(19) United States
(12) Patent Application Publication
(10) Pub. No.: US 2025/0221937 A1
(43) Pub. Date: Jul. 10, 2025

(54) STABLE PHARMACEUTICAL COMPOSITIONS COMPRISING ERDAFITINIB

(71) Applicant: Natco Pharma Limited, Banjara Hills, Hyderabad (IN)

(72) Inventors: Krishna Murthy Bhavanasi, Banjara Hills, Hyderabad (IN); Satyanarayana Vattikuti, Banjara Hills, Hyderabad (IN); Sundeep Mupparaju, Banjara Hills, Hyderabad (IN); Pavan Bhat, Banjara Hills, Hyderabad (IN); Venkaiah Chowdary Nannapaneni, Banjara Hills, Hyderabad (IN)

(21) Appl. No.: 18/853,465

(22) PCT Filed: Apr. 6, 2023
(86) PCT No.: PCT/IN2023/050335

§ 371 Date: Oct. 2, 2024

(30) Foreign Application Priority Data
Apr. 7, 2022 (IN) 202241012175

(51) Int. Cl. A61K 9/20; A61K 9/16; A61K 31/498

(52) U.S. Cl. CPC A61K 9/2018; A61K 9/1623; A61K 9/1652; A61K 9/2013; A61K 9/2059; A61K 31/498

(57) ABSTRACT

The present invention relates to stable pharmaceutical compositions comprising a kinase inhibitor. More particularly, the present invention relates to stable tablet compositions comprising erdafitinib and one or more pharmaceutically acceptable excipients and processes for preparing such compositions.

### FIELD OF THE INVENTION

The present invention relates to stable pharmaceutical compositions comprising a kinase inhibitor. More particularly, the present invention relates to stable tablet compositions comprising Erdafitinib and one or more pharmaceutically acceptable excipients and processes for preparing such compositions.

### BACKGROUND OF THE INVENTION

Erdafitinib is a kinase inhibitor and is described chemically as N-(3,5-dimethoxyphenyl)-N′-(1-methylethyl)-N-[3-(1-methyl-1H-pyrazol-4-yl) quinoxalin-6-yl]ethane-1,2-diamine.

Erdafitinib is approved as an anti-cancer agent in the form of tablets and marketed under the brand name BALVERSA® by Janssen Biotech Inc.

BALVERSA® is indicated for the treatment of adult patients with locally advanced or metastatic urothelial carcinoma (mUC) that are susceptible to FGFR3 or FGFR2 genetic alterations, and that have progressed during or following at least one line of prior platinum-containing chemotherapy.

U.S. Pat. No. 8,895,601 discloses a genus of quinoxaline derivative compounds and pharmaceutical compositions generally of these compounds. The broad genus of compounds disclosed in U.S. Pat. No. 8,895,601 includes erdafitinib.

U.S. Pat. No. 10,898,482 discloses pharmaceutical compositions comprising erdafitinib or a pharmaceutically acceptable salt or a solvate thereof, a formaldehyde scavenger, and a pharmaceutically acceptable carrier. In one aspect of U.S. Pat. No. 10,898,482, there is provided the use of a formaldehyde scavenger, and in particular meglumine, to increase the stability of erdafitinib or a pharmaceutically acceptable salt or a solvate thereof, and in particular erdafitinib free base, in a pharmaceutical composition, in particular a solid pharmaceutical composition, and more particularly a solid pharmaceutical composition in the form of a capsule or a tablet.

According to U.S. Pat. No. 10,898,482, it was found that erdafitinib is sensitive to degradation, especially when incorporated in a solid pharmaceutical composition. In particular, erdafitinib is sensitive, especially when incorporated in a solid pharmaceutical composition, to transformation into cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine. As such, U.S. Pat. No. 10,898,482 discloses that the stability of erdafitinib compositions can be increased using a formaldehyde scavenger, in particular meglumine.

This conversion decreases the stability of erdafitinib, especially when incorporated in a solid pharmaceutical composition, which reduces its bioavailability over time. As such, U.S. Pat. No. 10,898,482 discloses that the stability of erdafitinib pharmaceutical compositions can be increased by using a formaldehyde scavenger, in particular meglumine, in the pharmaceutical compositions.

However, there exists a need to develop alternative pharmaceutical compositions comprising erdafitinib, which are free of any formaldehyde scavengers and that are stable. In particular, there exists a need to develop an alternative pharmaceutical composition comprising erdafitinib in which erdafitinib is resistant to degradation, including degradation into cyclized products such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine.

### SUMMARY OF THE INVENTION

An objective of the present invention is to develop stable pharmaceutical compositions comprising erdafitinib and one or more pharmaceutically acceptable excipients. In particular, it is an object of the present invention to develop pharmaceutical compositions that are resistant to erdafitinib degradation, such as degradation into cyclized products such as 6,8-dimethoxy-4-(1-methylethyl)-1-[3-(1-methyl-1H-pyrazol-4-yl)quinoxaline-6-yl]-2,3,4,5-tetrahydro-1H-1,4-benzodiazepine.

The present invention relates to pharmaceutical compositions, including solid oral dosage forms, such as tablets and capsules, comprising erdafitinib and one or more pharmaceutically acceptable excipients that are stable. In particular, the pharmaceutical compositions of the present invention are resistant to erdafitinib degradation into degradation products, such as degradation into a cyclized product such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine.

The present invention also relates to processes for the preparation of stable pharmaceutical compositions, including solid oral dosage forms such as tablets and capsules, comprising erdafitinib and one or more pharmaceutically acceptable excipients having a comparable dissolution and content uniformity to the marketed dosage form BALVERSA® that are bioequivalent to BALVERSA®.

The present invention further provides stable pharmaceutical compositions, including solid oral dosage forms such as tablets and capsules, comprising erdafitinib and one or more pharmaceutically acceptable excipients, which are free of any formaldehyde scavenger. In particular, the pharmaceutical compositions of the present invention are resistant to erdafitinib degradation, such as degradation into a cyclized products such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, which are free of any formaldehyde scavenger such as meglumine.

In one embodiment, the present invention relates to stable tablet compositions comprising erdafitinib and one or more pharmaceutically acceptable excipients, which are free of any formaldehyde scavenger. In particular, the stable tablet compositions of the present invention are resistant to erdafitinib degradation, such as degradation into cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, which are free of any formaldehyde scavenger such as meglumine.

The present invention also relates to processes for preparing stable pharmaceutical compositions, including solid oral dosage forms, such as tablets and capsules, comprising erdafitinib and one or more pharmaceutically acceptable excipients, which are free of any formaldehyde scavenger. In particular, the processes provide stable pharmaceutical compositions that are resistant to erdafitinib degradation, such as degradation into cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, which are free of any formaldehyde scavenger such as meglumine.

In some embodiments the processes relate to stable tablet compositions comprising erdafitinib and one or more pharmaceutically acceptable excipients, which are free of any formaldehyde scavenger. In particular, the processes provide stable pharmaceutical compositions that are resistant to erdafitinib degradation, such as degradation into cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, which are free of any formaldehyde scavenger such as meglumine.

The present inventors have surprisingly discovered that pharmaceutical compositions comprising erdafitinib, which are free of any formaldehyde scavengers such as meglumine, are stable even after storage at elevated temperatures and high relative humidity, and that such stable compositions have comparable/better dissolution properties compared to the marketed erdafitinib tablet dosage form BALVERSA®.

The present inventors also have surprisingly discovered that pharmaceutical compositions having a substantially higher drug load for erdafitinib, compared with the prior art, unexpectedly provide erdafitinib compositions that are stable and, in particular, are resistant to erdafitinib degradation into cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, without the need for a formaldehyde scavenger, such as meglumine.

The present inventors have further discovered that pharmaceutical compositions having a substantially higher drug load for erdafitinib, compared with the prior art, and which and are free of any formaldehyde scavenger provide compositions having a substantially lower total weight than those disclosed in the prior art, which is expected to increase patient compliance. In some embodiments, the substantially higher drug load for erdafitinib is achieved by reducing the amounts of the one or more excipients contained in the formulation to increase the drug load while achieving a stable composition. In some embodiments, the inventors have discovered that the pharmaceutical compositions free of any formaldehyde scavenger can be made stable and, in particular, can be made resistant to erdafitinib degradation, including into cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, through the choice of the one or more excipients contained in the compositions.

### DETAILED DESCRIPTION OF THE INVENTION

The following is a detailed description of embodiments of the present invention. The embodiments are in such detail as to clearly communicate the invention. However, the amount of detail offered is not intended to limit the anticipated variations of embodiments; on the contrary, the intention is to cover all modifications, equivalents, and alternatives falling within the spirit and scope of the present invention as defined by the appended claims.

Unless the context requires otherwise, throughout the specification which follow, the word “comprise” and variations thereof, such as, “comprises” and “comprising” are to be construed in an open, inclusive sense that is as “including, but not limited to.” It is to be appreciated that the terms “comprising”, “comprises” and “comprised of” as used herein include, but are not limited to, the terms “consisting of”, “consistently essentially of,” “consists of,” “consists essentially of,” and “consists” within their meaning.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics of the present invention may be combined in any suitable manner to provide one or more embodiments of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing numerical quantities of ingredients, properties such as concentration, and so forth, used to describe and claim certain embodiments of the present invention are understood to be modified by the term “about,” e.g., an amount of about 10% by weight, an amount of about 98% by weight, an amount in the range of from about 10% to about 90% by weight. Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. Nonetheless, in some embodiments, the numerical values set forth in the specific examples disclosed herein are modified by the term “about.”

The recitation of ranges of values herein is intended to serve as a shorthand method of referring individually to each separate value falling within the range. Unless otherwise indicated herein, each individual value falling within the scope of a range is incorporated into the specification as if it were individually recited herein. For example, a range of 1-10 includes the values 1 and 10, as well as the values 9, 8, 7, 6, 5, 4, 3, 2 and any non-integer in between.

All processes and methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used in a claim is not defined below, it should be given the broadest definition persons in the pertinent art have given that term as reflected in printed publications and issued patents at the time of filing.

The term “active ingredient” or “active agent” or “drug” are interchangeable terms that refer to an active agent that induces a desired pharmacological or physiological effect.

The terms “formaldehyde scavenger” as used herein refers to compounds, other than erdafitinib, that are capable of absorbing formaldehyde. Formaldehyde scavengers include compounds comprising a nitrogen center that is reactive with formaldehyde, such as to form one or more reversible or irreversible bonds between the formaldehyde scavenger and formaldehyde. For example, formaldehyde scavengers comprise one or more nitrogen atoms/centers that are reactive with formaldehyde to form a Schiff base imine that is capable of subsequently binding with formaldehyde. For example, the formaldehyde scavenger comprises one or more nitrogen centers that are reactive with formaldehyde to form one or more 5-8 membered cyclic rings. A formaldehyde scavenger preferably comprises one or more amine or amide groups. For example, a formaldehyde scavenger can be an amino acid, an amino sugar, an alpha amine compound, or a conjugate or derivative thereof, or a mixture thereof. A formaldehyde scavenger may comprise two or more amines and/or amides.

Formaldehyde scavengers include, for example, glycine, alanine, serine, threonine, cysteine, valine, leucine, isoleucine, methionine, phenylalanine, tyrosine, aspartic acid, glutamic acid, arginine, lysine, ornithine, citrulline, taurine pyrrolysine, meglumine, histidine, aspartame, proline, tryptophan, citrulline, pyrrolysine, asparagine, glutamine, or a conjugate or mixture thereof; or, whenever possible, pharmaceutically acceptable salts thereof. In one embodiment, the formaldehyde scavenger can be meglumine. As described herein, the disclosed pharmaceutical compositions are free of these formaldehyde scavengers, including for example meglumine.

The term “pharmaceutically acceptable” as used herein means that which is useful in preparing a pharmaceutical composition that is generally considered safe and non-toxic.

The terms “solid dosage form” or “dosage form” or “composition” as used herein refer to a dosage form suitable for administration. “Solid dosage forms” and “solid oral dosage forms” refer to dosage forms that may be administered orally, such as tablets, capsules, spheroids, mini-tablets, pellets, granules, pills and the like. In certain embodiments these solid dosage forms are tablets or capsules and in particular embodiments, tablets.

The term “stable” as used herein means that the erdafitinib active ingredient is resistant to change in its purity or its concentration over time when stored. The term “resistant to degradation” means that the erdafitinib active ingredient is resistant to a change in its purity or its concentration over time based on the formation of degradation products formed through decomposition or reaction with other chemical compounds to form new compounds that are not the active ingredient.

A “stable pharmaceutical composition” or a “pharmaceutical composition that is stable” therefore refers to a pharmaceutical composition comprising erdafitinib that that is resistant to a change in the purity of erdafitinib or the concentration of erdafitinib in the composition over time. In some embodiments, a stable pharmaceutical composition comprising erdafitinib is stable because the erdafitinib contained therein is resistant to degradation into degradation products including cyclized products, such 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine.

A pharmaceutical composition comprising erdafitinib that is resistant to erdafitinib degradation into degradation products including cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine, is a composition that is resistant to a change in erdafitinib purity or erdafitinib concentration in the composition over time due to the formation of degradation products including cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine.

As disclosed herein, pharmaceutical compositions comprising erdafitinib are considered “stable” if the amount of erdafitinib initially contained in the composition, e.g., the labeled amount of erdafitinib in the composition (e.g., tablet), does not change by more than 10% over time, e.g., after a predetermined amount of time in storage under a predetermined set of storage conditions as described below. In preferred embodiments, pharmaceutical compositions comprising erdafitinib are considered stable if the amount of erdafitinib initially contained in the composition does not change by more than 5% over time, more preferably by more than 2% over time, and even more preferably by more than 1% over time. The amount of change is determined as a percent by weight based on the total weight of the pharmaceutical composition. When the pharmaceutical composition is a tablet, this weight % change is based on total uncoated tablet weight (even if the pharmaceutical composition is a coated tablet).

As disclosed herein, pharmaceutical compositions comprising erdafitinib are “resistant to erdafitinib degradation”, when they are resistant to erdafitinib degrading into degradation products such as cyclized products, such as 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine. Pharmaceutical compositions comprising erdafitinib are considered resistant to degradation if the amount of total amount of degradation products is not more than 2% by weight based on the total weight of erdafitinib weight in the composition, preferably not more than 1%, more preferably not more than 0.5%, and even more preferably not more than 0.1%, e.g., after a predetermined amount of time in storage under a predetermined set of storage conditions as described below.

The predetermined amount of time in storage under a predetermined set of storage conditions for resisting degradation and/or evaluating stability includes, but is not limited to, storage for: 3 months at 25° C.±2° C. and 40%±5% relative humidity, 6 months at 25° C.±2° C. and 40%±5% relative humidity, 12 months at 25° C.±2° C. and 40%±5% relative humidity, and 24 months at 25° C.±2° C. and 40%±5% relative humidity; storage for 1 month at 30° C.±2° C. and 65%±5% relative humidity, 2 months at 30° C.±2° C. and 65%±5% relative humidity, 3 months at 30° C.±2° C. and 65%±5% relative humidity, and 6 months at 30° C.±2° C. and 65%±5% relative humidity; and storage for 1 month at 40° C.±2° C. and 75%±5% relative humidity, 2 months at 40° C.±2° C. and 75%±5% relative humidity, 3 months at 40° C.±2° C. and 75%±5% relative humidity, and 6 months at 40° C.±2° C. and 75%±5% relative humidity.

In one embodiment, the shelf life for the stable erdafitinib composition is greater than three months at 40° C.±2° C. and 75±5% relative humidity and under these conditions, the labeled amount of erdafitinib in the composition (e.g., tablet), does not change by more than 10%. In certain embodiments, the erdafitinib compositions as disclosed herein the labeled amount of erdafitinib in the composition (e.g., tablet), does not change by more than 10% for 12 months at 25° C.±2° C. and 40%±5% relative humidity. In specific embodiments, the erdafitinib compositions as disclosed herein the labeled amount of erdafitinib in the composition (e.g., tablet), does not change by more than 10% for 24 months at 25° C.±2° C. and 40%±5% relative humidity.

Pharmaceutical compositions comprising erdafitinib are considered resistant to degradation if the amount of total amount of degradation products is not more than 2% by weight based on the total weight of erdafitinib weight in the composition, preferably not more than 1%, more preferably not more than 0.5%, and even more preferably not more than 0.1%.

In another embodiment, the shelf life for the stable erdafitinib composition that are resistant to degradation is greater than three months at 40° C.±2° C. and 75±5% relative humidity and under these conditions, the total amount of degradation products is not more than 2% by weight based on the total weight of erdafitinib weight in the composition, or not more than 1%, or not more than 0.5%, or not more than 0.1%. In certain embodiments, the above amounts of degradation products are achieved for 12 months at 25° C.±2° C. and 40%±5% relative humidity. In other embodiments, the above amounts of degradation products are achieved for 24 months at 25° C.±2° C. and 40%±5% relative humidity

As described herein, labeled amount of erdafitinib in the composition (e.g., tablet) and/or the amount of degradation products can be achieved under the above described times in storage under the described storage conditions even when the composition contains erdafitinib in an amount of from 3% to 6% w/w, or in an amount from 4% to 6% w/w based on total weight of the composition, all without an optional coating.

The term “erdafitinib” as used herein includes, but is not limited to, erdafitinib free base, and its pharmaceutically acceptable salts, solvates hydrates, enantiomers, isomers, ethers, esters, prodrugs, polymorphs and derivatives thereof. “Erdafitinib” according to the present invention may be in a crystalline form, an amorphous form or a mixture of both crystalline and amorphous forms. Preferably, erdafitinib is erdafitinib free base, or a solvate, hydrate, or polymorph thereof, or a pharmaceutically acceptable salt of erdafitinib or a solvate, hydrate, or polymorph thereof. Most preferably, erdafitinib is erdafitinib free base, or a solvate, hydrate, or polymorph thereof.

The phrase “free of any formaldehyde scavenger” means containing less than 0.1% by weight of any formaldehyde scavenger, preferably less than 0.05% by weight of any formaldehyde scavenger, more preferably less than 0.01% by weight of any formaldehyde scavenger. In certain embodiments “free of any formaldehyde scavenger(s)” means containing zero % by weight formaldehyde scavenger or no detectable amount (according to the limit of detection) of any formaldehyde scavenger.

As used in the specification and the appended claims, the singular forms “a”, “an”, and “the” include plural references unless the context clearly dictates otherwise. Thus for example, reference to “a method” includes one or more methods, and/or steps of the type described herein and/or which will become apparent to those persons skilled in the art upon reading this disclosure so forth.

The term “including” is used to mean “including but not limited to.” “Including” and “including but not limited to” are used interchangeably.

Ratios, concentrations, amounts, and other numerical data may be presented herein in a range format. It is to be understood that such range format is used merely for convenience and brevity and should be interpreted flexibly to include not only the numerical values explicitly recited as the upper and lower limits of the range, but also to include all the individual numerical values or sub-ranges encompassed within that range as if each numerical value and sub-range is explicitly recited. For example, a weight percentage range of 0.001 to 50% (w/w) should be interpreted to include not only the explicitly recited limits of 0.01% (w/w) to 50% (w/w) but also to include sub-ranges, such as 0.05% to 40% (w/w), 0.1% to 7% (w/w) and so forth, as well as individual amounts, including fractional amounts, within the specified ranges, such as 0.022%, 0.01%, 0.58%, 0.9%, 1.39%, 5.4%, 22.6% for example. In some embodiments, numerical ranges ratios, concentrations, amounts, and the like understood to be modified by the term “about.”

As used herein, the term “% w/w” refers to the percent by weight. When used in the context of a pharmaceutical composition of the present invention, “% w/w” refers to the percent by weight of a component of the composition, an active ingredient or an impurity, based on the total weight of the composition without an optional coating.

The term “about” is meant to designate any value which lies within the range of ±10%, preferably within a range of ±5%, more preferably within a range of ±2%, still more preferably within a range of ±1% of a stated numerical value or numerical range. For example, “about 10” should be construed as meaning within the range of 9 to 11, preferably within the range of 9.5 to 10.5, more preferably within the range of 9.8 to 10.2, and still more preferably within the range of 9.9 to 10.1.

“Pharmaceutically acceptable excipient(s)” are excipients that are “pharmaceutically acceptable” as defined above. Pharmaceutically acceptable excipient(s) are components added to a pharmaceutical composition to, for example, facilitate manufacture, enhance stability, control release, enhance product characteristics, enhance bioavailability, enhance patient acceptability, etc.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

In some embodiments, the present invention relates to a stable pharmaceutical composition comprising erdafitinib and one or more pharmaceutically acceptable excipients.

In some embodiments, the present invention relates to a process for the preparation of a stable pharmaceutical composition comprising erdafitinib and one or more pharmaceutically acceptable excipients, including the stable pharmaceutical compositions discussed in the foregoing embodiments.

In some embodiments, the present invention relates to a stable pharmaceutical composition comprising erdafitinib and one or more pharmaceutically acceptable excipients, wherein the composition is free of any formaldehyde scavengers.

In some embodiments, the present invention relates to a stable pharmaceutical composition comprising erdafitinib, an antioxidant and one or more pharmaceutically acceptable excipients.

In some embodiments, the present invention relates to a stable pharmaceutical composition comprising erdafitinib, an antioxidant and one or more pharmaceutically acceptable excipients, wherein the composition is free of any formaldehyde scavengers.

In some embodiments, the present invention relates to a process for the preparation of a stable tablet composition comprising erdafitinib and one or more pharmaceutically acceptable excipients, including the stable pharmaceutical compositions discussed in the foregoing embodiments.

In some embodiments, the present invention relates to a stable tablet composition comprising erdafitinib and one or more pharmaceutically acceptable excipients, wherein the composition is free of any formaldehyde scavengers.

In some embodiments, the present invention relates to a stable tablet composition comprising erdafitinib, an antioxidant and one or more pharmaceutically acceptable excipients.

In some embodiments, the present invention relates to a stable tablet composition comprising erdafitinib, an antioxidant and one or more pharmaceutically acceptable excipients, wherein the composition is free of any formaldehyde scavengers.

In another embodiment of the present invention, erdafitinib is present in a stable pharmaceutical composition in an amount from 0.1 to 40% w/w based on total weight of the composition without an optional coating. In some embodiments, erdafitinib is present in a stable pharmaceutical composition in an amount from 1% to 20% w/w, or in amount from 1% to 10% w/w, or in an amount from 2% to 6% w/w, or in an amount from 3% to 6% w/w, or in an amount from 4% to 6% w/w based on total weight of the composition, all without an optional coating.

In a preferred embodiment, the erdafitinib is present in a stable pharmaceutical composition in an amount greater than 3% w/w or greater than or equal to 4% w/w and less than or equal to 40% w/w based on total weight of the composition without an optional coating. In other embodiments the erdafitinib is present in an amount greater than 3% w/w or greater than or equal to 4% w/w and less than or equal to 20% w/w. In certain embodiments, the erdafitinib is present in an amount greater than 3% w/w or greater than or equal to 4% w/w and less than or equal to 10% w/w. In yet other embodiments, the erdafitinib is present in an amount of 3% w/w to 6% w/w or 4% w/w to 6% w/w.

In some embodiments, compositions according to the present invention comprise one or more additional pharmaceutically acceptable excipients selected from the group consisting of diluents/fillers, disintegrants/disintegrating agents, binders, surfactants, antioxidants, glidants and lubricants. These excipients may be present intragranularly, i.e., within granules in the composition, or extragranularly, i.e., outside of granules in the composition.

Diluents/fillers according to the present invention include, but are not limited to, lactose monohydrate, lactose anhydrous, fructose, dextrose, dextrates, dextrins, mannitol, lactitol, sorbitol, starch, sucrose, calcium carbonate, dibasic calcium phosphate, tribasic calcium phosphate, calcium sulfate, sodium chloride, microcrystalline cellulose, silicified microcrystalline cellulose, powdered cellulose, kaolin and the like or combinations thereof. In certain embodiments, the diluent can be present in a pharmaceutical composition of the invention in an amount from 50 to 98% based on total weight of the composition without an optional coating. In some embodiments, the amount of diluent is from 70% to 98% w/w, or from 80% to 97% w/w, or from 90% to 96% w/w, or from 90% to 94% w/w. In certain embodiments, the diluent is mannitol.

Binders according to the present invention include, but are not limited to, hydroxypropyl methylcellulose, hydroxypropyl cellulose, polyvinylpyrrolidone (povidone), gelatin, ethyl cellulose, polyvinyl alcohol, pregelatinized starch, carboxymethyl cellulose, sodium alginate, microcrystalline cellulose and the like or combinations thereof. In certain embodiments, the binder can be present in a pharmaceutical composition of the invention in an amount from 0 to 10% w/w based on total weight of the composition without an optional coating. In some embodiments, the binder is present in an amount from 0.1 to 10% w/w, or from 1 to 8% w/w, or from 2 to 7% w/w, or from 3 to 5% w/w.

Disintegrants/disintegrating agents according to the present invention include, but not limited to, starches or modified starches such as pregelatinized starch, partly pregelatinized starch; croscarmellose sodium, crospovidone, sodium starch glycolate, low substituted hydroxypropyl cellulose, hydroxypropyl cellulose, carmellose, carmellose calcium, carmellose sodium and the like or combinations thereof. In certain embodiments, the disintegrant can be present in an amount from 0.1 to 20% w/w. Preferably, the amount of disintegrant is from 0.5% to 10% w/w, or from 1% to 9% w/w, or from 1% to 5% w/w, or from 1% to 3% w/w. In certain embodiments, the disintegrant is sodium starch glycolate or croscarmellose sodium or a combination thereof.

Surfactants according to the present invention may be selected from anionic, cationic or non-ionic surface-active agents or surfactants. Suitable anionic surfactants include, but are not limited to, carboxylate, sulfonate, and sulfate ions such as sodium lauryl sulfate (SLS), sodium laurate, dialkyl sodium sulfosuccinates particularly bis-(2-ethylhexyl) sodium sulfosuccinate, sodium stearate, potassium stearate, sodium oleate and the like. Suitable cationic surfactants include, but are not limited to, those containing long chain cations, such as benzalkonium chloride, bis-2-hydroxyethyl oleyl amine or the like. Suitable non-ionic surfactants include, but are not limited to, polyoxyethylene sorbitan fatty acid esters (polysorbates), fatty alcohols such as lauryl, cetyl and stearyl alcohols; glyceryl esters such as the naturally occurring mono-, di-, and tri-glycerides; fatty acid esters of fatty alcohols; polyglycolized glycerides such as gelucire; polyoxyethylene-polyoxypropylene block co-polymer such as Poloxamer and other alcohols such as propylene glycol, polyethylene glycol. In certain embodiments, the surfactant can be present in a pharmaceutical composition of the invention in an amount from 0% to 10% w/w based on total weight of the composition without an optional coating. In some embodiments, the amount if from 0.1 to 10% w/w, or from 1% to 9% w/w, or from 3% to 7% w/w, or from 4% to 6% w/w.

Antioxidants according to the present invention include, but are not limited to, ascorbic acid, vitamin E, butylated hydroxytoluene (BHT), butylated hydroxyanisole (BHA), tartaric acid, citric acid, sodium metabisulfite, potassium metabisulfite, trivalent phosphorous, and divalent sulfur-containing compounds such as sulfides, thiodipropionates and organophosphites and the like, or combinations thereof. In certain embodiments, antioxidants can be present in a pharmaceutical composition of the invention in an amount from 0% to 5% w/w based on total weight of the composition without an optional coating. In some embodiments, the amount is from 0.1 to 5% w/w, or from 0.1% to 3% w/w, or from 0.1% to 2% w/w, or from 0.1% to 1% w/w. In certain embodiments, the antioxidant is sodium metabisulfite.

Lubricants according to the present invention include, but are not limited to, colloidal silicon dioxide, stearic acid, magnesium stearate, calcium stearate, zinc stearate, sodium stearyl fumarate, talc, hydrogenated castor oil, and mixtures thereof. In certain embodiments, lubricants can be present in a pharmaceutical composition of the invention in an amount from 0.1 to 5% w/w based on total weight of the composition without an optional coating. In some embodiments, the amount of lubricant is from 0.5 to 5% w/w, or from 0.5 to 4% w/w, or from 0.5 to 2% w/w, or from 1 to 1.5%. In certain embodiments, the lubricant is sodium stearyl fumarate.

Glidants according to the present invention include, but are not limited to, calcium silicate, magnesium silicate, magnesium trisilicate, talc, colloidal silicon dioxide and mixtures thereof mixtures thereof. In certain embodiments, the glidants can be present in a pharmaceutical composition of the invention in an amount from 0 to 5% w/w based on total weight of the composition without an optional coating. In some embodiments, the amount is 0.1 to 5% w/w, or from 0.5 to 5% w/w, or from 1 to 4% w/w, or from 2 to 3% w/w.

In one embodiment, the present invention relates to a stable pharmaceutical composition comprising:
(a) erdafitinib,
(b) one or more diluents,
(c) one or more disintegrants,
(d) one or more lubricants, and
(e) optionally one or more excipients selected from the group consisting of a binder, a surfactant, an antioxidant and a glidant,
   wherein the composition is free of any formaldehyde scavengers. The stable pharmaceutical composition is as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable pharmaceutical composition comprising:
(a) 1 to 20% w/w of Erdafitinib, preferably 4 to 6% w/w, and more preferably 2 to 5% w/w,
(b) 50 to 98% w/w of one or more diluents, preferably 80% to 97% w/w, and more preferably 90% to 96% w/w,
(c) 0.1 to 20% w/w of one or more disintegrants, preferably 1% to 5% w/w, and more preferably 1% to 3% w/w,
(d) 0.1 to 5% w/w of one or more lubricants, preferably 0.5 to 2% w/w, and more preferably from 1 to 1.5%, and
(e) optionally one or more excipients selected from the group consisting of 0.1% to 10% w/w of a binder, 0.1% to 10% w/w of a surfactant, 0.1% to 5% w/w of an antioxidant, and 0.1% to 5% w/w of a glidant,
   wherein the composition is free of any formaldehyde scavengers. Any of the weight percentage ranges disclosed for one components (a)-(e) may be combined with any of the above weight percentage ranges disclosed for another component (a)-(e). The stable pharmaceutical compositions are as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable pharmaceutical composition comprising:
(a) 1 to 20% w/w of Erdafitinib, preferably 4 to 6% w/w, and more preferably 2 to 5% w/w,
(b) 80 to 97% w/w of mannitol, preferably 90% to 96% w/w, and more preferably 90% to 94% w/w,
(c) 0.5 to 10% w/w of sodium starch glycolate or croscarmellose sodium or a combination thereof, preferably 1% to 5% w/w, and more preferably 1% to 3% w/w, and
(d) 0.5 to 5% w/w of sodium stearyl fumarate, preferably 0.5 to 2% w/w, and more preferably from 1 to 1.5%,
   wherein the composition is free of any formaldehyde scavengers. Any of the weight percentage ranges disclosed for one components (a)-(d) may be combined with any of the above weight percentage ranges disclosed for another component (a)-(d). The stable pharmaceutical compositions are as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable tablet composition comprising:
(a) 1 to 20% w/w of Erdafitinib, preferably 4 to 6% w/w, and more preferably 2 to 5% w/w,
(b) 80 to 95% w/w of mannitol, preferably 90% to 96% w/w, and more preferably 90% to 94% w/w,
(c) 0.5 to 10% w/w of sodium starch glycolate or croscarmellose sodium or a combination thereof, preferably 1% to 5% w/w, and more preferably 1% to 3% w/w, and
(d) 0.5 to 5% w/w of sodium stearyl fumarate, preferably 0.5 to 2% w/w, and more preferably from 1 to 1.5%,
   wherein the composition is free of any formaldehyde scavengers. Any of the weight percentage ranges disclosed for one components (a)-(d) may be combined with any of the above weight percentage ranges disclosed for another component (a)-(d). The stable pharmaceutical compositions are as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable pharmaceutical composition comprising:
(a) 1 to 10% w/w of Erdafitinib, preferably 4 to 6% w/w, and more preferably 2 to 5% w/w,
(b) 85 to 98% w/w of mannitol, preferably 90% to 96% w/w, and more preferably 90% to 94% w/w,
(c) 0.1 to 20% w/w of sodium starch glycolate or croscarmellose sodium or a combination thereof, preferably 1% to 5% w/w, and more preferably 1% to 3% w/w, and
(d) 0.1 to 5% w/w of sodium stearyl fumarate, preferably 0.5 to 2% w/w, and more preferably from 1 to 1.5%,
   wherein the composition is free of any formaldehyde scavengers. Any of the weight percentage ranges disclosed for one components (a)-(d) may be combined with any of the above weight percentage ranges disclosed for another component (a)-(d). The stable pharmaceutical compositions are as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable tablet composition comprising:
(a) 1 to 10% w/w of Erdafitinib, preferably 4 to 6% w/w, and more preferably 2 to 5% w/w,
(b) 85 to 98% w/w of mannitol, preferably 90% to 96% w/w, and more preferably 90% to 94% w/w,
(c) 0.1 to 20% w/w of sodium starch glycolate or croscarmellose sodium or a combination thereof, preferably 1% to 5% w/w, and more preferably 1% to 3% w/w, and
(d) 0.1 to 5% w/w of sodium stearyl fumarate, preferably 0.5 to 2% w/w, and more preferably from 1 to 1.5%,
   wherein the composition is free of any formaldehyde scavengers. Any of the weight percentage ranges disclosed for one components (a)-(d) may be combined with any of the above weight percentage ranges disclosed for another component (a)-(d). The stable pharmaceutical compositions are as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable tablet composition comprising:
(a) 1 to 20% w/w of Erdafitinib,
(b) 80 to 95% w/w of mannitol,
(c) 0.5 to 10% w/w of sodium starch glycolate,
(d) 0.5 to 5% w/w of sodium stearyl fumarate,
   wherein the composition is free of any formaldehyde scavengers.

In another embodiment, the present invention relates to a stable tablet composition comprising:
(a) 1.0-10.0 mg of erdafitinib, preferably 3.0-5.0 mg,
(b) 40.0-150.0 mg of mannitol, preferably 60.0-120.0 mg, and more preferably 100.0-120.0 mg
(c) 0.5-10.0 mg of sodium starch glycolate or croscarmellose sodium or a combination thereof, preferably 1.25-7.5 mg, and more preferably 1.5-3.5 mg, and
(d) 0.5.0-10.0 mg of sodium stearyl fumarate, preferably 0.5-5.0 mg, more preferably 0.5-2.5 mg,
   wherein the composition is free of any formaldehyde scavengers. Any of the milligram ranges disclosed for one components (a)-(d) may be combined with any of the above milligram ranges disclosed for another component (a)-(d). The stable pharmaceutical compositions are as defined above, meaning pharmaceutical compositions resistant to erdafitinib degradation into degradation products and/or contain a constant labeled amount of erdafitinib in the composition (e.g., tablet) as described above.

In another embodiment, the present invention relates to a stable tablet composition comprising:
(a) 1.0-10.0 mg of Erdafitinib,
(b) 50.0-150.0 mg of mannitol,
(c) 0.5-10.0 mg of sodium starch glycolate,
(d) 1.0-10.0 mg of sodium stearyl fumarate,
   wherein the composition is free of any formaldehyde scavengers.

In another embodiment, the present invention relates to a process for the preparation of a stable pharmaceutical composition, comprising the steps of:
(i) blending erdafitinib with one or more pharmaceutically acceptable excipients, and
(ii) formulating the blend of step (i) into suitable dosage form.

In another embodiment, the present invention relates to a process for the preparation of a stable tablet composition, comprising the steps of:
(i) blending erdafitinib with one or more pharmaceutically acceptable excipients, and
(ii) compressing the blend of step (i) into a tablet dosage form.

In another embodiment, the present invention relates to a process for the preparation of a stable tablet composition, comprising the steps of:
(i) blending erdafitinib with one or more pharmaceutically acceptable excipients,
(ii) lubricating the blend of step (i) with one or more lubricants, and
(iii) compressing the lubricated blend of step (ii) into tablet dosage form.

In another embodiment, the present invention relates to a process for the preparation of a stable tablet composition, comprising the steps of:
(i) blending erdafitinib with one or more pharmaceutically acceptable excipients,
(ii) lubricating the blend of step (i) with one or more lubricants,
(iii) compressing the lubricated blend of step (ii) into tablet dosage form, and
(iv) coating the tablets prepared in step (iii) with a coating composition. Coating compositions suitable for use according to the present invention are described below.

In another embodiment, the present invention relates to a process for the preparation of a stable tablet composition, comprising the steps of:
(i) blending erdafitinib with one or more pharmaceutically acceptable excipients,
(ii) granulating the blend of step (i),
(iii) blending the granules of step (ii) with one or more pharmaceutically acceptable excipients, and
(iv) compressing the blend of step (iii) into tablet dosage form.

In another embodiment, the present invention relates to a process for the preparation of a stable tablet composition, comprising the steps of:
(i) blending erdafitinib with one or more pharmaceutically acceptable excipients,
(ii) granulating the blend of step (i),
(iii) blending the granules of step (ii) with one or more pharmaceutically acceptable excipients,
(iv) compressing the blend of step (iii) into tablet dosage form, and
(v) coating the tablets prepared in step (iv) with a coating composition.

In another embodiment, the present invention relates to a process for the preparation of a stable tablet composition, comprising the steps of:
(i) blending erdafitinib with one or more pharmaceutically acceptable excipients,
(ii) granulating the blend of step (i),
(iii) blending the granules of step (ii) with one or more pharmaceutically acceptable excipients,
(iv) lubricating the blend of step (iii) with one or more lubricants and
(v) compressing the lubricated blend of step (iv) into tablet dosage form.

In another embodiment, the present invention relates to a process for the preparation of a stable tablet composition, comprising the steps of:
(i) blending erdafitinib with one or more pharmaceutically acceptable excipients,
(ii) granulating the blend of step (i),
(iii) blending the granules of step (ii) with one or more pharmaceutically acceptable excipients,
(iv) lubricating the blend of step (iii) with one or more lubricants and
(v) compressing the lubricated blend of step (iv) into tablet dosage form.
(vi) coating the tablets prepared in step (v) with a coating composition. In a preferred embodiment, the process provides a tablet composition that is stable and, in particular, that is resistant to erdafitinib degradation.

In another embodiment, the present invention relates to a process for the preparation of tablet composition, comprising the steps of:
(i) blending erdafitinib with a diluent, disintegrant and optionally a lubricant,
(ii) granulating the blend of step (i) by roller compaction,
(iii) blending the granules of step (ii) with one or more pharmaceutically acceptable excipients,
(iv) lubricating the blend of step (iii) with a lubricant, and
(v) compressing the blend of step (iv) into tablet dosage form. In a preferred embodiment, the process provides a tablet composition that is stable and, in particular, that is resistant to erdafitinib degradation.

In another embodiment, the present invention relates to a process for the preparation of a stable tablet composition, comprising the steps of:
(i) blending erdafitinib with a diluent, disintegrant and optionally a lubricant,
(ii) granulating the blend of step (i) by roller compaction,
(iii) blending the granules of step (ii) with one or more pharmaceutically acceptable excipients,
(iv) lubricating the blend of step (iii) with a lubricant, and
(v) compressing the blend of step (iv) into tablet dosage form.
(vi) coating the tablets prepared in step (v) with a coating composition.

In another embodiment, the present invention relates to a process for the preparation of a stable tablet composition, comprising the steps of:
(i) blending erdafitinib with mannitol, sodium starch glycolate and sodium stearyl fumarate,
(ii) granulating the blend of step (i) by roller compaction,
(iii) lubricating the granules of step (ii) with extragranular sodium stearyl fumarate, and
(iv) compressing the blend of step (iii) into tablet dosage form.

In another embodiment, the present invention relates to a process for the preparation of a stable tablet composition, comprising the steps of:
(i) blending erdafitinib with mannitol, sodium starch glycolate and sodium stearyl fumarate,
(ii) granulating the blend of step (i) by roller compaction,
(iii) lubricating the granules of step (ii) with extragranular sodium stearyl fumarate,
(iv) compressing the blend of step (iii) into tablet dosage form and
(v) coating the tablets prepared in step (iv) with a coating composition. In another embodiment, the pharmaceutical composition according to the present invention is in the form of tablets, capsules, granules, powder, pellets and sachets.

In another embodiment, the blend is formulated into a suitable dosage form like tablets or capsules using different techniques which are well known in the prior art.

In another embodiment, the compositions of the present invention may be prepared using any method known in the art, but are not limited to wet granulation, dry granulation, roller compaction, direct compression, melt granulation, solid dispersion, extrusion spherinization and encapsulation.

In another embodiment, the solvents used for granulation process may be selected from water, isopropyl alcohol, methanol, ethanol, methylene chloride or combination thereof.

The composition according to the present invention may be uncoated or optionally coated with functional coating, film coating, moisture barrier coating or a protective coating composition. The coating may be selected from amongst one or more of those suitable coating materials known in the art. Coating may be performed by applying one or more film forming polymers, with or without other pharmaceutically inert excipients, as a solution/suspension using any conventional coating technique known in the art, such as spray coating in a conventional coating pan or fluidized bed processor or dip coating.

The amount of the film coating may be 1 to 10% w/w, preferably, 1 to 3% w/w of the total composition. Any of a variety of film coatings can be used in the present composition. Suitable film coating may include, but are not limited to, polymers, plasticizers, pigments, opacifiers, glidants, binders, anti-tacking agents, antifoaming agents, surfactants, fillers, extenders, coloring agents and the like.

Examples of film-forming polymers include ethylcellulose, hydroxypropyl methylcellulose, polyvinyl alcohol (PVA), hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit® RL and RS; and the like. Alternatively, commercially available coating compositions comprising film forming polymers marketed under various trade names, such as Opadry® may also be used for coating the tablets. The coating can be obtained as a dry blend concentrate.

The film coating may also optionally include a plasticizer such as triacetin, propylene glycol, diethyl phthalate, tributyl sebacate, polyethylene glycol (PEG) or glceryl mono and dicaprylocaprate; a surfactant such as sodium lauryl sulphate, an anti-adherent or glidant such as talc, fumed silica or magnesium stearate; an opacifying agent such as titanium dioxide. Coloring agents may be selected from FDA approved colorants such as Iron Oxide, Lake of Tartrazine, Allura Red, Lake of Quinoline Yellow, Lake of Erythrosine, titanium dioxide and the like.

In another embodiment, the film coating composition according to the present invention is OPADRY® AMB II composition which contains polyvinyl alcohol-partially hydrolyzed, talc, titanium dioxide, glceryl mono and dicaprylocaprate, sodium lauryl sulfate and iron oxide.

In another embodiment, the coating according to the present invention is applied by solubilising or suspending the excipients in solvents such as isopropyl alcohol, water, acetone, ethanol, methylene chloride, hydrochloric acid and the like, or mixtures thereof.

In another embodiment, the pharmaceutical composition according to the present invention is in the form of tablets, and in some embodiments immediate release tablets.

In another embodiment, the pharmaceutical composition according to the present invention is in the form of tablets which may be coated or uncoated.

In another embodiment, the tablet according to the present invention may be round or oval. The edges of the tablets can be beveled or rounded. In another embodiment, the tablets are ovoid or round. The tablets according to the invention may be scored.

In another embodiment, the present invention provides a composition comprising erdafitinib in the range of 0.1 mg to 50 mg, preferably 1 mg to 20 mg, more preferably 1 mg to 10 mg, and even more preferably 3 mg to 5 mg (e.g., 3 mg, 4, mg, or 5 mg).

In another embodiment, the present invention provides a composition comprising erdafitinib in an amount of 3 mg to 5 mg.

In another embodiment of the present invention, the weight of the uncoated core tablet composition is in the range of about 50 mg to about 500 mg, preferably about 50 mg to about 300 mg and more preferably about 75 mg to about 125 mg.

In another embodiment of the present invention, the weight of the uncoated core tablet composition is in the range of about 75 mg to about 125 mg.

In another embodiment of the present invention, the weight ratio of erdafitinib to the uncoated core tablet is about 1:100, preferably about 1:50 and more preferably about 1:25.

In another embodiment of the present invention, the weight ratio of erdafitinib to the uncoated core tablet is 1:25

In another embodiment, the composition comprising erdafitinib according to the present invention can be used for the treatment of cancer by administering to patients in need of cancer therapy.

In another embodiment, the composition comprising erdafitinib according to the present invention can be used for the treatment of patients with locally advanced or metastatic urothelial carcinoma (mUC), that has susceptible FGFR3 or FGFR2 genetic alterations, and progressed during or following at least one line of prior platinum-containing chemotherapy.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1: Tablet Composition Comprising Erdafitinib

| | | Quantity per tablet |
|---|---|---|
| S. No | Ingredients | (% w/w) |
| 1 | Erdafitinib | 2-5% |
| 2 | Mannitol | 80-97% |
| 3 | Croscarmellose sodium | 1.0-5.0% |
| 4 | Sodium metabisulfite | 0.01-1% |
| 5 | Sodium stearyl fumarate | 0.5-2.0% |
| | Core Tablet weight | 100.00% |
| | Film Coating Composition | Q.S |

The processing steps involved in manufacturing the tablets comprising Erdafitinib are given below:
(i) erdafitinib, mannitol, croscarmellose sodium and sodium metabisulfite were sifted separately and blended,
(ii) the blend of step (i) was lubricated with sodium stearyl fumarate,
(iii) the lubricated blend of step (ii) was compressed into tablets, and
(iv) the tablets prepared in step (iii) were coated with film coating composition.

### Example 2: Tablet Composition Comprising Erdafitinib

| | | Quantity | Percent w/w for |
|---|---|---|---|
| S. No | Ingredients | (mg/tablet) | uncoated tablet |
| 1 | Erdafitinib | 5.00 | 4.0% |
| 2 | Mannitol | 115.00 | 92.0% |
| 3 | Sodium starch glycolate | 3.75 | 3.0% |
| 4 | Sodium stearyl fumarate | 1.25 | 1.0% |
| | Core Tablet weight | 125.00 | 100.0% |
| 5 | Opadry ® Film Coating | 5.00 | |
| | composition | | |
| | Coated Tablet Weight | 130.00 | |

The processing steps involved in manufacturing the tablets comprising Erdafitinib are given below:
(i) erdafitinib, mannitol and sodium starch glycolate were sifted separately and blended,
(ii) the blend of step (i) was lubricated with sodium stearyl fumarate,
(iii) the lubricated blend of step (ii) was compressed into tablets, and
(iv) the tablets prepared in step (iii) were coated with film coating composition.

### Example 3: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 3.00 | 6.0% |
| 2 | Mannitol | 45 | 90.0% |
| 3 | Croscarmellose sodium | 1.5 | 3.0% |
| 4 | Sodium stearyl fumarate | 0.5 | 1.0% |
| | Core Tablet weight | 50.00 | 100.0% |
| 6 | Opadry ® Film Coating | 2.00 | |
| | composition | | |
| | Coated Tablet Weight | 52.00 | |

The process involved in manufacturing the tablets of Example 3 is similar to the process provided in Example 2.

### Example 4: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 5.00 | 4.0% |
| 2 | Mannitol | 113.75 | 91.0% |
| 3 | Sodium starch glycolate | 3.75 | 3.0% |
| 4 | Sodium stearyl fumarate | 0.625 | 0.50% |
| | Extragranular components | | |
| 5 | Sodium stearyl fumarate | 1.875 | 1.50% |
| | Core Tablet weight | 125.00 | 100.0% |
| 6 | Opadry ® Film Coating | 5.00 | |
| | composition | | |
| | Coated Tablet Weight | 130.00 | |

The processing steps involved in manufacturing the tablets comprising erdafitinib are given below:
(i) erdafitinib, mannitol, sodium starch glycolate and sodium stearyl fumarate were sifted separately and blended,
(ii) the blend of step (i) was compacted using roller compaction and milled,
(iii) the milled granules obtained in step (ii) were lubricated with extragranular sodium stearyl fumarate,
(iv) the lubricated blend of step (iii) was compressed into tablets, and
(v) the tablets prepared in step (iv) were coated with film coating composition.

### Example 5: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 5.00 | 4.0% |
| 2 | Mannitol | 116.25 | 93.0% |
| 3 | Sodium starch glycolate | 1.25 | 1.0% |
| 4 | Sodium stearyl fumarate | 0.625 | 0.50% |
| | Extragranular components | | |
| 5 | Sodium stearyl fumarate | 1.875 | 1.50% |
| | Core Tablet weight | 125.00 | 100.0% |
| 6 | Opadry ® Film Coating | 5.00 | |
| | composition | | |
| | Coated Tablet Weight | 130.00 | |

### Example 6: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 5.00 | 2.0% |
| 2 | Dicalcium phosphate | 235.0 | 94.0% |
| 3 | Croscarmellose sodium | 7.5 | 3.0% |
| 4 | Sodium stearyl fumarate | 0.625 | 0.250% |
| | Extragranular components | | |
| 5 | Sodium stearyl fumarate | 1.875 | 0.750% |
| | Core Tablet weight | 250.00 | 100.0% |
| 6 | Opadry ® Film Coating | 10.00 | |
| | composition | | |
| | Coated Tablet Weight | 260.00 | |

### Example 7: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 5.00 | 2.0% |
| 2 | Mannitol | 235 | 94.0% |
| 3 | Croscarmellose sodium | 7.5 | 3.0% |
| 4 | Sodium stearyl fumarate | 0.625 | 0.250% |
| | Extragranular components | | |
| 5 | Sodium stearyl fumarate | 1.875 | 0.750% |
| | Core Tablet weight | 250.00 | 100.0% |
| 6 | Opadry ® Film Coating | 10.00 | |
| | composition | | |
| | Coated Tablet Weight | 260.00 | |

The process involved in manufacturing the tablets of Examples 5-7 is similar to the process provided in Example 4.

### Example 8: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 3.00 | 4.0% |
| 2 | Mannitol | 68.250 | 91.0% |
| 3 | Sodium starch glycolate | 2.250 | 3.0% |
| 4 | Sodium stearyl fumarate | 0.375 | 0.50% |
| | Extragranular components | | |
| 5 | Sodium stearyl fumarate | 1.125 | 1.50% |
| | Core Tablet weight | 75.00 | 100.0% |
| 6 | Opadry ® Film Coating | 3.00 | |
| | composition | | |
| | Coated Tablet Weight | 78.00 | |

The processing steps involved in manufacturing the tablets comprising erdafitinib are given below:
(i) erdafitinib, mannitol, sodium starch glycolate and sodium stearyl fumarate were sifted separately and blended,
(ii) the blend of step (i) was compacted using roller compaction and milled,
(iii) the milled granules obtained in step (ii) were lubricated with extragranular sodium stearyl fumarate,
(iv) the lubricated blend of step (iii) was compressed into tablets, and
(v) the tablets prepared in step (iv) were coated with film coating composition.

### Example 9: Tablet Composition Comprising Erdafitinib

| | | Quantity | Percent w/w for |
|---|---|---|---|
| S. No | Ingredients | (mg/tablet) | uncoated tablet |
| | Intragranular components | | |
| 1 | Erdafitinib | 4.00 | 4.0% |
| 2 | Mannitol | 91.00 | 91.0% |
| 3 | Sodium starch glycolate | 3.00 | 3.0% |
| 4 | Sodium stearyl fumarate | 0.500 | 0.50% |
| | Extragranular components | | |
| 5 | Sodium stearyl fumarate | 1.500 | 1.50% |
| | Core Tablet weight | 100.00 | 100.0% |
| 6 | Opadry ® Film Coating | 4.00 | |
| | composition | | |
| | Coated Tablet Weight | 104.00 | |

The processing steps involved in manufacturing the tablets comprising erdafitinib are given below:
(i) erdafitinib, mannitol, sodium starch glycolate and sodium stearyl fumarate were sifted separately and blended,
(ii) the blend of step (i) was compacted using roller compaction and milled,
(iii) the milled granules obtained in step (ii) were lubricated with extragranular sodium stearyl fumarate,
(iv) the lubricated blend of step (iii) was compressed into tablets, and
(v) the tablets prepared in step (iv) were coated with film coating composition.

### Dissolution Data

Table 1 given below provides comparative dissolution profile of erdafitinib tablets 5 mg prepared according to Examples 4 and 5 in comparison with BALVERSA® (erdafitinib) tablets 5 mg in 500 ml of 0.01 N HCl (pH 2.0) as dissolution medium, in USP I apparatus (Basket) at 75 rpm.

TABLE 1
Comparative dissolution profile of erdafitinib
tablets 5 mg prepared according to Examples 4
and 5 with BALVERSA ® (erdafitinib) tablets 5 mg

| | Cumulative % drug released | | |
|---|---|---|---|
| Time | BALVERSA ® (erdafitinib) | | |
| (in min) | tablets 5 mg | Example - 4 | Example -5 |
| 5 | 95.00 | 101.00 | 97.00 |
| 10 | 96.00 | 101.00 | 99.00 |
| 15 | 97.00 | 100.00 | 99.00 |
| 20 | 97.00 | 100.00 | 100.00 |
| 30 | 97.00 | 100.00 | 100.00 |

Based on the above data more than 85% release was observed within 15 minutes in both erdafitinib tablets 5 mg prepared according to Examples 4 and 5 and BALVERSA® (erdafitinib) tablets 5 mg. Hence, it was concluded that the dissolution profile of erdafitinib tablets 5 mg prepared according to Examples 4 and 5 was comparable with BALVERSA® (erdafitinib) tablets 5 mg.

### Stability Data

Table 2 below provides results for 3 months of stability data for the 5 mg erdafitinib tablets of Example 4. The tablets were stored at 40° C. and 75% relative humidity for three months. Samples were analyzed by HPLC to verify that retention time of the major peak in the chromatograph of the samples corresponded to the chromatogram of the standard preparation for erdafinitib as obtained in the assay.

TABLE 2
Water content, % erdafitinib, and total degradation product
were assessed at one, two, and three months. The percentage
of erdafitinib is compared against the labeled amount.

| | Stability Study Compilation Data, 5 mg batch | | | |
|---|---|---|---|---|
| | (40° C. ± 2° C./75% ± 5% RH) | | | |
| | | 1^{st} | 2^{nd} | 3^{rd} |
| Characteristic | Initial | Month | Month | Month |
| Water Content | 0.3% | 0.4% | 0.5% | 0.6% |
| Erdafitinib | 101.0% | 98.6% | 99.8% | 99.3% |
| (% w/w by HPLC) | | | | |
| Total Degradation | 0.10% | 0.05% | 0.10% | 0.05% |
| Products | | | | |

What is claimed is:

1. A stable pharmaceutical composition comprising erdafitinib and one or more pharmaceutically acceptable excipients, wherein the composition is free of any formaldehyde scavengers, and wherein the erdafitinib content in the pharmaceutical composition does not change by more than 10% by weight based on the total weight of an uncoated composition, when stored under conditions selected from the group consisting of 12 months at 25° C.±2° C. and 40%±5% relative humidity, 24 months at 25° C.±2° C. and 40%±5% relative humidity, and 3 months at 40° C.±2° C. and 75%±5% relative humidity.

2. The stable pharmaceutical composition of claim 1 wherein the condition is 3 months at 40° C.±2° C. and 75%±5% relative humidity.

3. The stable pharmaceutical composition of claim 1 wherein the erdafitinib content in the pharmaceutical composition does not change by more than 5% by weight.

4. The stable pharmaceutical composition of claim 1 wherein the erdafitinib content in the pharmaceutical composition does not change by more than 2% by weight.

5. The stable pharmaceutical composition of claim 1 wherein the erdafitinib content in the pharmaceutical composition does not change by more than 1% by weight.

6. A stable pharmaceutical composition as claimed in claim 1 wherein the composition is in the form of a tablet, capsules, granules, powder, pellets, and sachets.

7. A stable pharmaceutical composition as claimed in claim 1 wherein one or more pharmaceutically acceptable excipients are selected from diluents, disintegrants, binders, surfactants, antioxidants, glidants, and lubricants.

8. A stable pharmaceutical composition as claimed in claim 1 wherein the composition is prepared by a method selected from wet granulation, dry granulation, roller compaction, direct compression, melt granulation, solid dispersion, extrusion spheronization, and encapsulation.

9. A stable pharmaceutical composition as claimed in claim 1 wherein the composition is in the form a tablet and the weight ratio of erdafitinib to the uncoated core tablet is 1:25.

10. A stable pharmaceutical composition comprising:
a. 1% to 20% w/w of erdafitinib, preferably 4% to 6% w/w, and more preferably 2% to 5% w/w;
b. 50% to 98% w/w of one or more diluents, preferably 80% to 97% w/w, and more preferably 90% to 96% w/w;
c. 0.1% to 20% w/w of one or more disintegrants, preferably 1% to 5% w/w, and more preferably 1% to 3% w/w;
d. 0.1% to 5% w/w of one or more lubricants, preferably 0.5% to 2% w/w, and more preferably from 1% to 1.5% w/w; and
e. optionally one or more excipients selected from the group consisting of 0.1% to 10% w/w of a binder, 0.1% to 10% w/w of a surfactant, 0.1% to 5% w/w of an antioxidant, and 0.1% to 5% w/w of a glidant;
wherein the composition is free of any formaldehyde scavengers.)

11. A stable pharmaceutical composition according to claim 10 comprising:
a. 1% to 20% w/w of erdafitinib, preferably 4% to 6% w/w, and more preferably 2% to 5% w/w;
b. 80% to 97% w/w of mannitol, preferably 90% to 96% w/w, and more preferably 90% to 94% w/w;
c. 0.5% to 10% w/w of sodium starch glycolate or croscarmellose sodium or a combination thereof, preferably 1% to 5% w/w, and more preferably 1% to 3% w/w; and
d. 0.5% to 5% w/w of sodium stearyl fumarate, preferably 0.5% to 2% w/w, and more preferably from 1% to 1.5% w/w;
wherein the composition is free of any formaldehyde scavengers.

12. A stable pharmaceutical composition as claimed in claim 11 comprising:
a. 1% to 20% w/w of erdafitinib;
b. 80% to 95% w/w of mannitol;
c. 0.5% to 10% w/w of sodium starch glycolate; and
d. 0.5% to 5% w/w of sodium stearyl fumarate;
wherein the composition is free of any formaldehyde scavengers.

13. A stable tablet composition according to claim 1 comprising:
a. 1.0 mg-10.0 mg of erdafitinib;
b. 50.0 mg-150.0 mg of mannitol;
c. 0.5 mg-10.0 mg of sodium starch glycolate; and
d. 1.0 mg-10.0 mg of sodium stearyl fumarate;
wherein the composition is free of any formaldehyde scavengers.

14. A process for the preparation of tablet composition, comprising the steps of:
i. blending erdafitinib with one or more pharmaceutically acceptable excipients;
ii. granulating the blend of step (i);
iii. blending the granules of step (ii) with one or more pharmaceutically acceptable excipients; and
iv. compressing the blend of step (iii) into tablet dosage form.

15. A process for the preparation of tablet composition as claimed in claim 14 comprising the steps of:
i. blending erdafitinib with a diluent, disintegrant, and optionally a lubricant;
ii. granulating the blend of step (i) by roller compaction;
iii. blending the granules of step (ii) with one or more pharmaceutically acceptable excipients;
iv. lubricating the blend of step (iii) with a lubricant; and
v. compressing the blend of step (iv) into tablet dosage form.

16. A process for the preparation of tablet composition as claimed in claim 15 comprising the steps of:
i. blending erdafitinib with mannitol, sodium starch glycolate, and sodium stearyl fumarate;
ii. granulating the blend of step (i) by roller compaction;
iii. lubricating the granules of step (ii) with extragranular sodium stearyl fumarate; and
iv. compressing the blend of step (iii) into tablet dosage form.
